(19) United States
(12) Patent Application Publication
(10) Pub. No.: US 2006/0122095 A1
(43) Pub. Date: Jun. 8, 2006

(54) Mucus formulation for mucosal surfaces and uses thereof

(76) Inventors: Brid Delvin, Berwyn, PA (US); Roger L Schnaare, Huntingdon, PA (US)

Correspondence Address: MORGAN LEWIS & BOCKIUS LLP, 1111 PENNSYLVANIA AVENUE NW, WASHINGTON, DC, 20004, US

(21) Appl. No.: 10/527,690

(22) PCT Filed: Sep. 12, 2003
(86) PCT No.: PCT/US03/28582

§ 371 Date: Jul. 27, 2005

Related U.S. Application Data
(60) Provisional application No. 60/409,983, filed on Sep. 12, 2002.
(60) Provisional application No. 60/458,949, filed on Apr. 1, 2003.

(51) Int. Cl. A61K 31/573; A61K 31/70; A61K 31/19; A61K 38/38

(52) U.S. Cl. 514/2; 514/23; 514/557; 514/179

(57) ABSTRACT

The present invention includes formulations comprising synthetic vaginal fluid, synthetic cervical mucus, and, a mixture of a synthetic cervical mucus and synthetic vaginal fluid suitable for mucosal administration of at least one therapeutic agent. The formulations of the invention are useful in the treatment, prevention and control of diseases and conditions in a subject in need thereof. The present invention also includes methods of administering the formulations. Also included are methods of using the formulations in the treatment, prevention and control of diseases or conditions affecting other mucus membranes, such as the nose, throat and gastrointestinal tract.

### CROSS-REFERENCE TO RELATED APPLICATIONS

This application claims priority to U.S. Provisional Application No. 60/409,983, filed Sep. 12, 2002 and U.S. Provisional Application No. 60/458,949, filed Apr. 1, 2003, both herein incorporated by reference in their entirety.

### FIELD OF THE INVENTION

The present invention generally pertains to a drug delivery system for mucosal administration of agents for treatment, prevention or control of diseases or conditions of a subject in need thereof. The present invention also pertains to methods for the prevention and control of sexually transmitted and other diseases using synthetic formulations comprising at least one therapeutic agent having the ability to inactivate pathogens and/or treat, prevent or control a disease. The present invention includes formulations useful for drug delivery into or through mucosal surfaces.

### BACKGROUND OF THE INVENTION

The vagina is lined with stratified squamous epithelium; therefore, it is unable to produce mucus. However, mucus and secretions from the endometrial and salpingeal mucous membranes collect in the vagina (Barnhart, K. and Shalaby, W., The Vagina: Physiological Characteristics Important to Formulators of Microbicides, Vaginal Microbicide Formulations Workshop, Ed. William F. Rencher, Lippincott-Raven Publishers, PA (1998), pp. 1-15). Additionally, a serum transudate known commonly as vaginal fluid also collects in the vagina. Cervical mucus is a viscous fluid composed of mucin glycoproteins, plasma proteins, and other proteins (e.g. lactoferrin), enzymes, amino acids, cholesterol, lipids and a range of inorganic ions, the concentrations of which fluctuate during the menstrual cycle (Marriott, C. and Gregory, N. P., Mucus Physiology and Pathology, Bioadhesive Drug Delivery Systems, Eds. Vincent Lenaerts and Robert Gurny, CRC Press (1990), pp. 1-24).

The major component of mucus (90-99%) is water (Katz, D. F. and Dunmire, E. N., Cervical Mucus—Problems and Opportunities for Drug Delivery via the Vagina and Cervix, Advanced Drug Delivery Reviews 11: 385-401 (1993)). It is common knowledge that mucin makes up between 0.5 to 5% of mucus by weight and it is believed that the glycoproteins are responsible for the viscous nature of mucus. It has been reported that approximately 2-5 ml of cervical mucus and approximately 1 ml of vaginal fluid are present in the vaginal cavity at any given time (Barnhart and Shalaby, 1998). The large surface area of the vagina holds this material without leakage with the exception of a discharge, which occurs at the ovulation stage of the cycle. However, during sexual arousal, the total volume of fluid can increase two- to three-fold (Barnhart and Shalaby, 1998).

Evidence suggests that uterine contractions transport material into the upper vaginal tract within minutes of administration (Barnhart et al., Distribution of a Spermicide Containing Nonoxynol-9 in the Vaginal Canal and the Upper Female Reproductive Tract, Human Reproduction 16(6): 1151-54 (2001)). It is believed that this process is mediated by uterine peristalsis controlled by endocrine and possibly paracrine events (Kunz et al., Sonographic Evidence for the Involvement of the Utero-Ovarian Counter-Current System in the Ovarian Control of Directed Uterine Sperm Transport, Human Reproduction Update 4: 667-72 (1996)). Given this evidence, it is anticipated that any vaginal formulation will be transported up through the vagina; however, a fluid-like material or formulation without sufficient viscosity will leak from the vagina, an observation seen with many conventional vaginal formulations. A formulation that has the properties of synthetic cervical mucus/vaginal fluid should maximize its residence and adherence to the vaginal lining much like normal fluids.

UC-781 (N-[4-chloro-3-(3-methyl-2-butenyloxy)phenyl]) is a small (molecular weight=335) organic molecule of the carboxanilide class of compounds. The structure of UC-781 is shown below. It has been comprehensively evaluated as a non-nucleotide reverse transcriptase inhibitor of HIV-1 reverse transcriptase (RT), functioning by means of an irreversible binding to the RT (Borkow, G. and Parniak, M. A., Anti-HIV-1 Microbicide Potential of the Tight Binding Nonnucleoside Reverse Transcriptase Inhibitor UC781. AID Science 1(12) (2001). Online publication: http://adscience.org/Articles/adiscience010.asp.

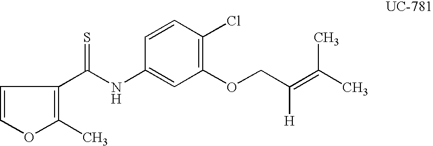

UC-781 is extremely hydrophobic, and can penetrate the viral envelope and core so as to bind to the RT, thus functioning as a virucidal agent. Its inhibition effect is extremely potent; inactivating primary wild type isolates of clades A through G at nanomolar concentrations in vitro. UC-781 is equally effective against strains of HIV-1 that encode known NNRTI resistance mutations. Importantly, UC-781 will inactivate virus produced by chronically infected cells and will also protect uninfected cells from subsequent infection. This “memory effect” was demonstrated by pre-treatment of susceptible cells with UC-781, followed by complete washing away of unbound drug, and then attempting infection (Borkow, G., Barnard, J., Nguyen, T. M., et al., Chemical barriers to human immunodeficiency virus type 1 (HIV-1) infection: Retrovirucidal activity of UC-781, a thiocarboxanilide nonnucleoside inhibitor of HIV-1 reverse transcriptase. J. Virol. 71:3023-3030 (1997)). In these studies, treated cells remained resistant to infection for several days. UC-781 had no toxic effect on different cell types in culture, even at extremely high concentrations. Studies designed to determine the absorption of UC-781 through multiple routes of administration were conducted in different species of animals and demonstrated that UC-781 has poor bioavailability. Thus, its potential use as a systenic agent is limited.

Burruano (Burruano, Brid, “In vitro Test to Evaluate the Interaction Between Synthetic Cervical Mucus and Vaginal Formulations,” Dissertation (2002)) developed a synthetic cervical mucus formulation that mimics the properties of fresh human mucus as per published literature (Burruano synthetic cervical mucus formulation (% w/w): guar gum, 1%; dried porcine gastric mucin (type III), 0.50%; imidurea, 0.30%; methylparaben, 0.15%, propylparaben, 0.02%; dibasic potassium phosphate, 0.26%; monobasic potassium phosphate, 1.57%; water, 96.20%). Previously published synthetic formulations are mostly polymeric solutions that provide viscosity but lack the mucin component of fresh mucus. A review of the literature suggests that the use of about 1% guar gum crosslinked with borate provides a reasonable viscosity level. Formulation development systematically evaluated guar gum (Hercules vs. Sigma), method of gum hydration, pH and presence of mucin (dried porcine gastric) for effect on viscosity and rheological flow; and, sodium azide, dimethylol-dimethylhydantoin, methylparaben, propylparaben and imidurea for their ability to function as preservatives. Results showed that the Hercules and Sigma guar gum yielded apparent viscosities of 8,083 and 5,983 cps at a shear rate of 1.50^{s-1}, respectively, at the same concentration, both within the range of 5,000 to 10,000 cps reported for mid-cycle human cervical mucus (Karni et al., Newtonian Viscosity of the Human Cervical Mucus During the Menstrual Cycle, Int. J. Fertil. 16: 185-8 (1971)).

The addition of mucin (0.01 to 1.0% w/w) exhibited an unanticipated change in rheological flow. Formulations containing up to 0.25% mucin exhibited pseudoplastic flow while at concentrations above this point dilatancy was observed. No single preservative was effective in protecting the formulation from microbial challenge, however, a combination of parabens and imidurea proved effective. The addition of the phosphate buffer achieved a pH of 7.4, similar to that reported for human cervical mucus (Tang et al., Comparison of Human Cervical Mucus and Sperm Media, Human Reproduction 14(11): 2812 (1999)), as well as add to the electrolyte content necessary for the formulation to be isotonic. The formulated synthetic cervical mucus formulation generated an osmolality of 272±6 mOsm/kg, which was well within the generally accepted tolerance of human mucosal tissue. Thus, guar gum crosslinked with borate containing 0.5% mucin and having a suitable preservative system provides a synthetic cervical mucus formulation with comparable viscosity, spinnbarkeit, and pH to that reported for human cervical mucus. The development of a distinct vaginal fluid simulant has previously been reported (Owen et al., A Vaginal Fluid Simulant, Contraception 59: 91-95 (1999)).

Typical vaginal formulations such as gels and creams have a tendency to leak out. Due to minimal leakage of vaginal fluids, compared to the volume produced, it is possible that a formulation that imitates resident fluids will leak less than conventional dosage forms, thereby enhancing the capacity for drug delivery with such a formulation.

Therefore, a need continues to exist for the development of a formulation that will readily mix with endogenous fluids, imitate resident mucosal fluids, and efficiently deliver therapeutic agents active against pathogens such viruses, other disease causing microbial cells, and deliver therapeutic agents active against other non-viral, non-microbial diseases such as, for example, cancer.

### SUMMARY OF THE INVENTION

The invention includes a method for delivering at least one therapeutic agent to a patient in need thereof comprising contacting patient mucosa with a formulation comprising: a) a composition selected from the group consisting of synthetic cervical mucus, synthetic vaginal fluid, and both synthetic cervical mucus and synthetic vaginal fluid, and, b) at least one therapeutic agent. In a highly preferred embodiment, the patient is human and the UC-781 is present in the formulation from at least about 0.001% to at least about 1.0% wt %.

The invention as also comprises a method for treating or preventing a disease comprising contacting mucosa of a patient in need thereof with a formulation comprising: a) a composition selected from the group consisting of: a composition comprising synthetic cervical mucus, a composition comprising synthetic vaginal fluid, and a composition comprising both synthetic cervical mucus and synthetic vaginal fluid, and, b) an amount of at least one therapeutic agent effective to treat or prevent the disease.

In another aspect of the invention, the invention includes a method for delivery of an effective amount of at least one therapeutic agent to a mucosal surface of a subject comprising administering a formulation to the mucosal surface wherein the formulation comprises guar gum present at about 1.00% w/w, dried gastric mucin (type III) present at about 0.50% w/w and a therapeutic agent present in an amount sufficient to be effective when the formulation is administered.

In another aspect of the invention, the invention also includes a method of producing a synthetic fluid composition wherein the synthetic fluid composition comprises properties substantially identical to naturally occurring vaginal fluid, the method comprising adding synthetic cervical mucus in an effective amount to a synthetic vaginal fluid to alter properties of the synthetic vaginal fluid to those substantially identical to the naturally occurring vaginal fluid.

The invention also includes a formulation comprising: a) a composition selected from the group consisting of synthetic cervical mucus; synthetic vaginal fluid; and synthetic cervical mucus and synthetic vaginal fluid; and, b) at least one therapeutic agent.

### BRIEF DESCRIPTION OF THE DRAWINGS

Not Applicable.

### DETAILED DESCRIPTION OF THE PREFERRED EMBODIMENTS

Definitions

As is generally the case in biotechnology and chemistry, the description of the present invention has required the use of a number of terms of art. Although it is not practical to do so exhaustively, definitions for some of these terms are provided here for ease of reference. Unless defined otherwise, all technical and scientific terms used herein have the same meaning as commonly understood by one of ordinary skill in the art to which this invention belongs. Definitions for other terms also appear elsewhere herein. However, the definitions provided here and elsewhere herein should always be considered in determining the intended scope and meaning of the defined terms. Although any methods and materials similar or equivalent to those described herein can be used in the practice of the present invention, the preferred methods and materials are described.

As used herein, the terms “anesthetic agent” or “anesthetic” means any drug which provides numbness and/or analgesia. A local anesthetic agent provides local numbness and/or analgesia. The term also includes, but is not limited to, any drug which, when locally administered, e.g., topically or by infiltration or injection, provides localized full or partial inhibition of sensory perception and/or motor function. Under either definition, the localized condition so induced is also referred to herein as “local anesthesia.” Local anesthetic agents which can be used include, simply by way of example, bupivacaine, ropivacaine, dibucaine, procaine, chloroprocaine, prilocaine, mepivacaine, etidocaine, tetracaine, lidocaine, and xylocaine, as well as anesthetically active derivatives, analogs and mixtures thereof. The local anesthetic can be in the form of a salt, for example, the hydrochloride, bromide, acetate, citrate, carbonate or sulfate. The local anesthetic agent can also be in the form of a free base. The term “local anesthetic” may also encompass a drug of a different class than those traditionally associated with local anesthetic properties, including but not limited to morphine, fentanyl, and agents which, for example, can provide regional blockade of nociceptive pathways (afferent and/or efferent). See, for example, U.S. Pat. No. 5,942,241.

As used herein, the term “bioadhesion” refers to an interfacial phenomenon in which two materials, at least one of which is biological, are held together by means of interfacial forces (Gandhi and Robinson, (1988)). For a polymer that adheres to the mucin layer of mucosal tissue, the term “mucoadhesion” is used. Bioadhesion and mucoadhesion may be measured using any assay method known in the art, such as, for example, the colloidal gold-mucin conjugate method (Tur and Ch'ng et al., (1998)); tensile testing (Nunez et al., (2000)); the fluorescent tube method (Park and Robinson, (1984)); and, rheological methods (Riley et al., (2001)), all of which are incorporated herein by reference in their entirety.

As used herein, the term “bioavailability” refers to the degree to which, or rate at which, a therapeutic agent or other substance becomes available at the site of physiological activity after administration.

As used herein, the term “derivative of an agent” refers to a chemically modified compound wherein the chemical modification takes place at one or more functional groups of the compound and/or on an aromatic ring, when present. The derivative however is expected to retain the pharmacological activity of the compound from which it is derived.

As used herein, the term “immunologic agent” refers to any therapeutic agent useful in the treatment of immunologically related diseases, such as for examples, cancer and non-malignant diseases or conditions.

As used herein, the term “microbe” is intended to include bacteria, fungi, yeast or protozoans which do not normally reside in the host and/or which are capable of causing host pathology.

As used herein, the term “microbicidal” means capable of inactivating or destroying microbes.

As used herein the term “mucus” or “mucin” refers to a composition which lines the respiratory, intestinal (including rectum) and reproductive tracts. It has several functions including protection, lubrication, and transport. Mucus is comprised of mucin glycoproteins that are highly glycosylated, high molecular weight molecules. The human mucin glycoproteins are encoded by a family of genes that share specific tandem repeats. Mucin glycoprotein is believed to be responsible for the gel-like structure of mucus. Mucin has a polypeptide backbone with covalently linked oligosaccharides side-chains and terminal sialic acids. The oligosaccharides are primarily attached to the polypeptide via O-linked bonds to serine and threonine. Cervical mucus is composed of mucin glycoproteins, plasma proteins other proteins such as lactoferrin, enzymes, amino acids, cholesterol, lipids and a range of inorganic ions, the concentrations of which fluctuate during the menstrual cycle. The major component of mucus (90-99%) is water. Mucin glycoprotein is believed to make up between about 0.5 to about 5% of mucus by weight. As used herein, the synthetic cervical mucus has a viscosity which can be optimized for delivery of a therapeutic agent. Of course, the optimal viscosity may depend upon the type of therapeutic agent to be delivered.

As used herein, the term “mucosa” refers to the layer(s) of cells lining the respiratory, intestinal (including rectum) and respiratory tracts.

As used herein, the term “patient” or “subject” broadly refers to any animal that is to be treated with the compositions and by the methods herein disclosed. The methods and compositions of the invention will find use in veterinary practice and animal husbandry wherever treatment or prevention of a disease or condition is convenient or desirable. In a preferred embodiment, the term includes humans in need of or desiring prevention or treatment of a disease or condition.

As used herein, the term “pharmaceutically acceptable” means that the ingredient is compatible with the other ingredients of the formulation and not injurious to the patient. Several pharmaceutically acceptable ingredients are known in the art and official publications such as THE UNITED STATES PHARMACOPEIA describe the analytical criteria used to assess the pharmaceutical acceptability of numerous ingredients of interest. The phrase “pharmaceutically acceptable” is used herein to mean that the material so described can be used for treatments in or on humans or other mammals without causing ill effects, such as toxicity, blistering or whitening of mucosal tissues.

As used herein, the term “rheological flow” refers to the spreading of the synthetic formulation on a mucosal surface.

As used herein, the term “spinnbarkeit” refers to a ductility property, and is independent of viscosity. Spinnbarkeit is a measure of the rheological properties of the mucus. Spinnbarkeit may be determined by placing a synthetic mucus preparation between thumb and forefinger and measuring the distance it took for the spindle to break.

As used herein, the term “surface tension” refers to a property of liquids arising from unbalanced molecular cohesive forces at or near the surface, as a result of which the surface tends to contract and has properties resembling those of a stretched elastic membrane.

As used herein, the term “therapeutic agent” includes any known pharmacologically active agent, such as for example, antibacterial agents, antiseptic agents, antibiotic agents, anti-inflammatory agents, antiparasitic agents, antiprotozoal agents, antiviral agents, antifungal agents and mixtures thereof. The term also includes hormones, analgesics and anesthetic agents and mixtures thereof. The term also includes any known pharmacologically active agent useful in the treatment or prevention of disease, i.e, such as a cancer of the gastrointestinal or reproductive tracts. The term also includes the pharmaceutically acceptable salt, prodrug such as an ester or an ether, or a salt of a prodrug, or a solvate such as ethanolae, or other derivative of such pharmacologically active agent. These salts, prodrugs, salts of prodrugs, solvates and derivatives are well-known in the art. The agent may be any of those which are approved for or used for the treatment, prophylaxis, cure or mitigation of any disease of the vulva, vagina, urinary tract, cervix or other female reproductive organ, or for any disease of the intestinal tract such as the rectum or colon, for diagnostic purposes, and, for systemic drug therapy. The agent must have utility when administered by delivery to all or a portion of the target mucosal surface(s). Therapeutic active agents maybe well-known agents due to their need for governmental approval or common usage or may be experimental agents in clinical trials or other experimental protocols. Many therapeutic agents are known in the art. See, for example, Remington: The Science and Practice of Pharmacy, 1995, Mack Publishing Co., Easton, Pa.

As used herein, the term “topical administration” refers to administration to the surface of the target mucosal tissue.

As used herein, the term “vaginal fluid simulant” refers to a synthetic fluid having a composition similar to that of naturally occurring vaginal fluid. One such vaginal fluid simulant developed in the art is that of Owen et al., 1999, having the following composition per liter: 3.51 gNaCl; 1.40 g KOH; 0.222 g Ca(OH)₂, 0.018 g bovine serum albumin; 2.00 g lactic acid; 1.00 g acetic acid, 0.16 g glycerol; 0.4 g urea, and, 5.0 g glucose. Other vaginal simulant fluids are known in the art. See, Owen et al., 1999. As used herein, the synthetic vaginal fluid has a viscosity which can be optimized for delivery of a therapeutic agent. Of course, the optimal viscosity may depend upon the type of therapeutic agent to be delivered.

As used herein, the term “virucidal” means capable of inactivating or destroying a virus.

As used herein, the term “viscosity” is defined as the ratio of the shear stress applied to the material, divided by the rate of shear. Sheer stress (dynes/cm²) is the force per unit area that is taken directly from the viscometer. The shear rate (units of^{s-1}) is obtained by multiplying the shear rate constant (k) by the rotation speed of the cone (RPM). Materials of a higher viscosity have a higher resistance to flow, or to forces which can induce flow, than a lower viscosity material. Rheologic characteristics given herein can be measured in any viscometer known in the art, such as, for example, a Brookfield viscometer. Use of 1% guar gum crosslinked with borate provides a reasonable viscosity level. However, use of any cross-linking agent in the amount necessary to result in a desired viscosity level is envisioned in the practice of the invention. Many other cross-linkers are known in the art.

The present invention includes a formulation which mixes with endogenous fluids of mucosal surfaces, particularly vaginal and rectal mucosa, and more efficiently delivers therapeutic agents. The formulation is readily miscible with vaginal or rectal fluids, and thus promoting rapid distribution of the therapeutic agent or agents, is maintained intravaginally or intrarectally in the same manner as the resident fluids thus ensuring maximal coverage of susceptible tissue with the therapeutic agent or agents and shows reduced potential expulsion or leakage of the dosage form, thereby maintaining a desired level of therapeutic agent.

The invention includes a method for delivering at least one therapeutic agent to a patient in need thereof comprising contacting patient mucosa with a formulation comprising: a) a composition selected from the group consisting of synthetic cervical mucus, synthetic vaginal fluid, and both synthetic cervical mucus and synthetic vaginal fluid, and, b) at least one therapeutic agent. In one embodiment, the mucosa is vaginal or rectal. In a preferred embodiment, the patient is human and the therapeutic agent is N-[4-chloro-3-(3-methyl-2-butenyloxy)phenyl] (UC-781) or a derivative thereof. In a highly preferred embodiment, the patient is human and the UC-781 is present in the formulation from at least about 0.001% to at least about 1.0% wt %. In another embodiment of the method, at least one therapeutic agent is selected from the group consisting of hormones, anti-microbial agents, anti-viral agents, analgesic agents and anaesthetic agents. In another embodiment of the method, the formulation is topically administered.

In a different embodiment of the method, the composition in step a) comprises the synthetic mucus and the synthetic vaginal fluid in a ratio which is optimal for delivery of at least one therapeutic agent. In highly preferred embodiment of the method, the synthetic vaginal fluid has at least two properties equal to, or substantially identical to, the properties of a composition comprising: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension. In another embodiment, the synthetic vaginal fluid has the following composition: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of 4.2.

In another aspect of the invention, the composition in step a) is synthetic cervical mucus comprising a viscosity which is optimal for delivery of at least one therapeutic agent. In another embodiment of the method, the viscosity of the synthetic cervical mucus is from about 2,000 cP to about 10,000 cP.

In one embodiment, the synthetic cervical mucus comprises guar gum present at about 1.00% w/w; and, dried gastric mucin (type III) present at about 0.50% w/w. In another embodiment, the synthetic cervical mucus further comprises imidurea, present at about 0.30% w/w; methylparaben, present at about 0.15% w/w; and, propylparaben, present at about 0.02% w/w. In a preferred embodiment, the synthetic cervical mucus further comprises dibasic potassium phosphate, present at about 0.26% w/w; and, monobasic potassium phosphate, present at about 1.57% w/w.

The invention also comprises a method for treating or preventing a disease comprising contacting mucosa of a patient in need thereof with a formulation comprising: a) a composition selected from the group consisting of: a composition comprising synthetic cervical mucus, a composition comprising synthetic vaginal fluid, and a composition comprising both synthetic cervical mucus and synthetic vaginal fluid, and, b) an amount of at least one therapeutic agent effective to treat or prevent the disease. In one embodiment of the invention, the mucosa is vaginal or rectal. In another aspect of the invention, the patient is human and the therapeutic agent is N-[4-chloro-3-(3-methyl-2-butenyloxy)phenyl] (UC-781) or a derivative thereof. In a highly preferred embodiment, the UC-781 is present in the formulation from at least about 0.1% to at least about 1.0% wt %. In a different embodiment of the invention, the at least one therapeutic agent is selected from the group consisting of hormones, anti-microbial agents, anti-viral agents, analgesic agents and anaesthetic agents. In another embodiment, the formulation is topically administered.

In a different aspect of the invention, the composition in step a) comprises the synthetic mucus and the synthetic fluid in a ratio which is optimal for delivery of at least one therapeutic agent. In a highly preferred embodiment of the invention, the synthetic vaginal fluid has at least two properties equal to, or substantially identical to, the properties of a composition comprising: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension.

In another embodiment, the synthetic vaginal fluid has the following composition: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of 4.2. In a different embodiment, the composition in step a) is synthetic cervical mucus comprising a viscosity which is optimal for delivery of at least one therapeutic agent. In a different embodiment of the method, the viscosity of the synthetic cervical mucus is from about 2,000 cP to about 10,000 cP.

In a preferred embodiment, the synthetic cervical mucus comprises guar gum present at about 1.00% w/w; and, dried gastric mucin (type III) present at about 0.50% w/w. In a highly preferred aspect, the synthetic cervical mucus further comprises imidurea, present at about 0.30% w/w; methylparaben, present at about 0.15% w/w; and, propylparaben, present at about 0.02% w/w. In a highly preferred embodiment, the synthetic cervical mucus further comprises dibasic potassium phosphate, present at about 0.26% w/w; and, monobasic potassium phosphate, present at about 1.57% w/w.

In another aspect of the invention, the invention includes a method for delivery of an effective amount of at least one therapeutic agent to a mucosal surface of a subject comprising administering a formulation to the mucosal surface wherein the formulation comprises guar gum present at about 1.00% w/w, dried gastric mucin (type III) present at about 0.50% w/w and a therapeutic agent present in an amount sufficient to be effective when the formulation is administered. In one embodiment, the mucosal surface is vaginal or rectal. In another embodiment, the formulation further comprises imidurea, present at about 0.30% w/w; methylparaben, present at about 0.15% w/w; and, propylparaben, present at about 0.02% w/w. In a preferred embodiment, the formulation further comprises dibasic potassium phosphate, present at about 0.26% w/w; and, monobasic potassium phosphate, present at about 1.57% w/w. In another preferred embodiment, the subject is human and the therapeutic agent is N-[4-chloro-3-(3-methyl-2 butenyloxy)phenyl] (UC-781) or a derivative thereof. In a highly preferred embodiment, the UC-781 is present in the formulation from at least about 0.001% to at least about 1.0% wt %. In a different embodiment, at least one therapeutic agent is selected from the group consisting of hormones, anti-microbial agents, anti-viral agents, analgesic agents and anaesthetic agents. In another embodiment, the formulation is topically administered. In a different embodiment, the formulation further comprises synthetic vaginal fluid in an amount sufficient for optimal delivery by the formulation of at least one therapeutic agent. In a preferred embodiment, the synthetic vaginal fluid has at least two properties equal to, or substantially identical to, the properties of a composition comprising: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension. In a highly preferred embodiment, the synthetic vaginal fluid has the following composition: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of 4.2.

In another aspect of the invention, the invention also includes a method of producing a synthetic fluid composition wherein the synthetic fluid composition comprises properties substantially identical to naturally occurring vaginal fluid, the method comprising adding synthetic cervical mucus in an effective amount to a synthetic vaginal fluid to alter properties of the synthetic vaginal fluid to those substantially identical to the naturally occurring vaginal fluid. In one embodiment, the properties of the naturally occurring vaginal fluid are selected from the group consisting pH, osmolarity and surface tension. In another embodiment, the synthetic fluid composition further comprises at least one therapeutic agent. In a preferred embodiment, the therapeutic agent is N-[4-chloro-3-(3-methyl-2-butenyloxy)phenyl] (UC-781) or a derivative thereof.

The invention also includes a formulation comprising: a) a composition selected from the group consisting of synthetic cervical mucus, synthetic vaginal fluid; and synthetic cervical mucus and synthetic vaginal fluid; and, b) at least one therapeutic agent. In one embodiment, at least one pharmaceutical agent is selected from the group consisting of hormones, anti-microbial agents, anti-viral agents, analgesic agents and anaesthetic agents. In another embodiment, the at least one therapeutic agent is UC-781 or a derivative thereof. In a different embodiment, the viscosity of the synthetic cervical mucus is from about 2,000 cP to about 10,000 cP.

In a highly preferred embodiment, the synthetic cervical mucus comprises guar gum present at about 1.00% w/w; and, dried gastric mucin (type III) present at about 0.50% w/w. In one embodiment, the synthetic cervical mucus further comprises imidurea, present at about 0.30% w/w; methylparaben, present at about 0.15% w/w; and, propylparaben, present at about 0.02% w/w. In a preferred embodiment, the synthetic cervical mucus further comprises dibasic potassium phosphate, present at about 0.26% w/w; and, monobasic potassium phosphate, present at about 1.57% w/w. In a highly preferred embodiment, the synthetic vaginal fluid has at least two properties equal to, or substantially identical to, the properties of a composition comprising: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension. In another highly preferred embodiment, the synthetic vaginal fluid has the following composition: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of 4.2.

Therapeutic Agents

Forms: Therapeutic agents useful in the practice of the invention include any known pharmacologically active agent as well as its pharmaceutically acceptable salt, prodrug such as an ester or an ether, or a salt of a prodrug, or a solvate such as ethanolae, or other derivative of such pharmacologically active agent.

Salts of the pharmacologically active agents may be derived from inorganic or organic acids and bases. Non-limiting examples of inorganic acids include hydrochloric, hydrobromic, sulfuric, nitric, perchloric, and phosphoric acids. Examples of bases include alkali metal (e.g., sodium) hydroxides, alkaline earth metal (e.g., magnesium) hydroxides, and ammonia. Other non-limiting examples of organic salts include: acetate, propionate, butyrate hexanoate, heptanoate, undecanoate, palmoate, cyclopentanepropionate, adipate, alginate, aspartate, benzoate, citrate, oxalate, succinate, tartarate, lactate, maleate, fumarate, camphorate, nicotinate, pectinate, picrate, pivalate, tosylate, gluconate, digluconate, hemisulfate, methanesulfonate, ethanesulfonate, 2-hydroxyethanesulfonate, dodecylsulfate, camphorsulfonate, benzenesulfonate, 2-naphthalenesulfonate, thiocyanate, phosphate glycerophosphate, and phenylpropionate. Other officially approved salts envisioned for use in the instant invention are listed in REMINGTON, supra.

In addition, this invention contemplates the use of polymorphic, isomeric (including stereoisomeric, geometrically isomeric and optically isomeric) and anomeric forms of the therapeutic agents described herein.

Types of Agents Delivered

The formulations of the present invention can be used to deliver practically any therapeutic agent. Accordingly, a wide variety of drugs encompassing a broad spectrum of therapeutic agents are contemplated as candidates for both local and or systemic delivery. The choice of a particular therapeutic agent or combination of agents depends merely on the treatment or application desired. Examples of these agents are well-known in the art. See, for example, REMINGTON, supra.

Non-limiting examples of pharmacological classes of drugs that can be used in the present formulations include: analgesics, anti-inflammatory agents, both of steroidal and non-steroidal nature, antihistamines, antipruritics, general and local anesthetics, mucolytics, mucoprotectants, antineoplastics, immunologic agents, antibiotics, antivirals, antidiabetics, uterine and antimigraine drugs, sedatives and hormones.

Analgesics include, but are not limited to, opiate and non-opiate analgesics and antagonists of both synthetic and natural origin. Some examples are: morphine derivatives, codeine dervatives, methadone, propoxyphene, meperidine, fentanyl, morphinans such as levorphanol, and pentazocine. Other analgesics include acetaminophen.

Non-limiting examples of non-steroidal anti-inflammatory agents contemplated as used in the invention include: propionic acids such as fenoprofen, ibuprofen, ketoprofen; fenamates such as meclofenamate and mefenamic acid; acetic acids such as diclofenac, etodolac, indomethacin, sulindac; oxicams such as piroxicam; and other agents such as nabumetone and oxyphenbutazone. Additionally, the following agents are also known as analgesic/anti-inflammatory agents: salicylates such as aspirin, methyl salicylate; monoglycol salicylate; and, salsalate.

Non-limiting examples of steroidal anti-inflammatory agents envisioned in the practice of the invention include hydrocortisone, prednisolone, dexamethasone, triamcinolone, fluocinolone, methylprednisolone, betamethasone, flumetasone, fluorometholone, beclomethasone, and fluocinonide.

Non-limiting examples of antihistamines include H₁ or H₂ antagonists or other types of histamine release inhibitors. The H₁ antagonists can be sedating or non-sedating. Examples of H₁-sedating antihistamines include diphenhydramine, chlorpheniramine, tripelennamine, promethazine, clemastine, and, doxylamine. Examples of H₁-non-sedating antihistamines include astemizole, terfenadine, and, loratadine. Examples of H₂ antagonists include cimetadine, famotidine, nizatidine, and ranitidine. Examples of histamine-release-inhibitors include cromolyn.

Non-limiting examples of vasoconstrictors include naphazoline, tetrahydrozoline, oxymetazoline, and, phenylephrine.

Examples of antibacterials include, but are not limited to, sulfa drugs, penicillins, cephalosporins, tetracyclines, erythromycins, aminoglycosides, polypeptide antibiotics, fluoroquinolones, chloramphenicol, clindamycin, rifampin, spectinomycin, vancomycin, bacitracin, cyclosporine, dapsone, ethambutol, ethionamide, isoniazid, nitrofurantoin, pyrazinamide, and trimethoprim.

Non-limiting examples of antiviral drugs include viral DNA polymerase inhibitors such as foscarnet, protease inhibitors, thymidine kinase inhibitors, sugar or glycoprotein synthesis inhibitors, structural protein synthesis inhibitors, attachment and adsorption inhibitors, amantadine, reverse transcriptase inhibitors, and nucleoside analogues such as acyclovir, didanosine, ganciclovir, idoxuridine, ribavarin, trifluridine, vidarabine, zalcitabine, zidovudine, acyclovir, penciclovir, valacyclovir, and ganciclovir.

Examples of mucolytics include, but are not limited to, potassium iodide, sodium thiocyanate, urea, guanidine hydrochloride, N-acetylcysteine, dithiotheritol, and proteolytic enzymes such as chymotrypsin and trypsin. These agents can be used to affect mucus production and the elasticity and viscosity of the mucus produced.

Non-liming examples of hormones include insulin, LHRH, growth hormone, calcitonin, thyroid hormones, and male and female hormones such as testosterones, estrogens and progesterones.

Examples of topical antifungals include, but are not limited to, haloprogin, ciclopirox, flucytosine, miconazole, econazole, clotrimazole, fluconazole, oxiconazole, sulconazole, metronidazole, itraconazole, ketoconazole, butaconazole, terconazole, nystatin, povidone-iodine, tolnaftate, benzoic acid, salicylic acid, mercuric oxide, resorcinol, triacetin, undecylenic acid and its calcium, copper and zinc salts.

Some topical anesthetics useful in the practice of the invention include, but are not limited to, benzyl alcohol, camphor, camphorated metacresol, juniper tar, menthol, phenol, phenolate sodium, resorcinol, methyl salicylate, turpentine oil, camphor, menthol, methyl nicotinate, capasaicin, capsicum containing capsaicin, capsicum oleoresin containing capsaicin. Other nonlimiting examples of local anesthetics useful in the practice of the invention include dibucaine, lidocaine, benzocaine, p-butylaminobenzoic acid-2-(diethylamino) ethyl ester, procaine, tetracaine, chloroprocaine, oxyprocaine, mepivacaine, bupivacaine, cocaine, piperocaine, and, dyclonine.

Non-limiting examples of topical bactericides and disinfectants include thimerosal, phenol, thymol, benzalkonium chloride, benzethonium chloride, chlorhexidine, povidone iodine, cetylpyridinium chloride, eugenol, and, trimethylammonium bromide.

Preservatives

Typical preservatives known for topical administration to mucosal surfaces include, but are not limited to, alcohol, ascorbyl palmitate, benzoic acid, butylated hydroxyanisole, butylated hydroxytoluene, chlorobutanol, ethylenediamine, ethylparaben, ethyl vanillin, glycerin, methylparaben, monothioglycerol, phenol, phenylethyl alcohol, phenylmercuric nitrate, propylparaben, sassafras oil, sodium benzoate, sodium formaldehyde sulfoxylate, sodium metabisulfite, sorbic acid, sulfur dioxide, maleic acid, and propyl gallate. Obviously, to the extent any of the foregoing preservatives are irritating to the target mucosal surface, less irritating preservatives should be chosen. See, for example, U.S. Pat. No. 6,017,521.

Furthermore, conventional and well known pharmaceutical excipients may be used including emulsifiers, suspending agents, osmotic enhancers, fragrances, preservatives and colors.

Application of Therapeutic Agents

The formulations can be introduced into the vaginal or rectal cavity, or other mucosal surfaces, by the use of conventional applicators such as tampon injectables or other coating or impregnating means. These formulations, unlike other systems, are not characterized by offensive leakage from the vaginal cavity following the insertion of the pharmaceutical composition. The volume administered will be from about 2 to 5 milliliters. Greater amounts may cause leakage and lesser amounts may not be effective to provide therapeutic levels of materials. Administration to the rectum is by methods known in the art.

The compositions release the therapeutic agent in the vaginal cavity or rectum due to the spreading of the formulation comprising the active agent or agents. Factors which effect the bioavailability of therapeutic agents are the components percentage of active agent; pH of the composition; and, diffusibility of the active species. Within the physiological environment of the vaginal cavity or rectum, all of the chemical and physical forces present, including fluids, enzymes, pH, chemical balance, temperature, and shear forces from body movement and body activity affect the rate of spreading of the formulation.

The manner of administration to the mucosa will preferably be designed to obtain direct contact of the formulations of the invention with sexually transmitted or disease causing microbes and/or viruses. The manner of administration for treatment of other diseases, such as a cancer, for example, is determined based on the disease and optimized accordingly. The topical composition may be prophylactically or therapeutically applied to human or other animal mucus membranes for the prevention and treatment of various medical conditions.

Bioavailability

Bioavailability is an absolute term that indicates measurement of both the true rate and total amount (extent) of therapeutic agent that reaches the target site from an administered dosage form. The physical characteristics of the therapeutic agent which affect and influence its bioavailability are physical parameters such as crystal form, choice of salt form, particle size, use of the hydrated or anhydrous form, wettability and solubility. Assessment of bioavailability is carried out by techniques and protocol known to those of skill in the art. See, for example, Remington, supra.

Topical Application

For topical applications, the formulations of the invention may additionally comprise organic solvents, emulsifiers, geling agents, moisturizers, stabilizers, other surfactants, wetting agents, preservatives, time release agents, and minor amounts of humectants, sequestering agents, dyes, perfumes, and other components commonly employed in pharmaceutical compositions for topical administration.

Diseases to be Treated or Prevented

The methods and compositions of the invention can be used to prevent and/or treat a broad spectrum of infections by pathogenic bacteria, viruses and other microbial organisms. A broad variety of microorganisms can potentially enter the vaginal tract or rectum through sexual intercourse, and result in a sexually transmitted disease. These microorganisms include bacteria, viruses, fungi, protozoa, and yeasts among others. The methods and formulations of the invention can be used to treat a wide variety of diseases, such as the STDs and other diseases and conditions, discussed below.

The abbreviation STD (sexually transmitted diseases) includes more than twenty specific organisms and syndromes, including HIV, Chlamydia Trachomatis, Genital Herpes, Genital Warts and Cervical Neoplasia. These include Acquired Immunodeficiency Syndrome (AIDS), Acute Urethral Syndrome or Cystitis, Bacterial Vaginosis Vulvovaginitis, Candidiasis, Cervical Intraepithelial Neoplasia, Chancroid, Chlamydia, Cytomegalovirus infections, Enteric infections, Genital Warts, Gonorrhea, Granuloma Inguinale, Hepatitis B, Herpes Genitalis, Human Papillomavirus (HPV), ALymphogranuloma venereum (LGV), Molluscum Contagiosum, Mucopurulent Cervicitis, Nongonococcal Urethritis, Pelvic Inflammatory Disease (PID), Syphilis, Trichomoniasis and Vulvovaginitis.

Other known STDs are Acquired Immunodeficiency Syndrome, caused by Human Immunodeficiency Virus (HIV); Acute Urethral Syndrome, caused by E. coli, C. trachomatis, N. gonorrhea and other gram-negative bacteria. Cervical Intraepithelial Neoplasia (CIN) has been associated with human papilloma virus (HPV), and the Herpes Simplex Virus. Chancroid is caused by Hemophilus Ducreyi. Chlamydia, one of the most common bacterial STD infections, is caused by Chlamydia trachomatis. Cytomegalovirus infections are caused by a DNA virus of the Herpes virus group. Genital Warts are caused by the human papillomavirus (HPV), a small DNA virus belong to the papillomavirus group. Gonorrhea is caused by Neisseria Gonorrhea, a gram-negative diplococcus. Granuloma Inguinale is caused by the gram-negative bacteria calymmato-bacterium granuloinatis. Hepatitis B is caused by Hepatatis B virus (HBV), a DNA virus with multiple antigenic components. Herpes Genitalis is caused by the Herpes Simplex II virus (HSV).

Lymphogranuloma venereum (LGV) is caused by Chlamydia Trachomatis. Molluscum Contagiosum is caused by the Molluscum Contagiosum virus, the largest DNA virus of the poxvirus group. Mucopurulent cervicitis is caused by Chlamydia and Gonorrhea. Nongonococcal Urethritis (NGU) is caused by Chlamydia of the D to K immunotypes. Pelvic Inflammatory Disease (PID) is caused by Gonorrhea, Chlamydia, and other anaerobic bacteria and gram-negative rods, such as E. coli and Mycoplasma homines. Syphilis is caused by Treponema pallidum, a spirochete. Vulvovaginitis is caused by Trichomonas vaginalis. The foregoing list is not intended to be in any way limiting of the diseases or conditions treatable or preventable by the methods, formulations and compositions of the invention. Other diseases related to mucosal surfaces, such as the rectum, for example, are treatable or preventable by the methods and formulations and compositions of the invention.

The following examples are illustrative of preferred embodiments of the invention and are not to be construed as limiting the invention thereto. All percentages are based on the percent by weight of the final delivery system or formulation prepared unless otherwise indicated and all totals equal 100% by weight.

### EXAMPLES

### Example 1

The invention includes a new type of vaginal formulation that is mixed with endogenous fluids and more efficiently delivers active agents. To test this, a microbicide formulation that mimics the resident fluids of the vagina is investigated. This formulation would differ from conventional vaginal gel or cream formulations in that its physical properties would be essentially identical to the fluids found in vivo. Such a formulation would be readily miscible with vaginal fluids promoting rapid distribution of the drug, be maintained in the rugae of the vagina in the same manner as resident fluids ensuring maximal coverage of susceptible tissue with the drug, and reduce potential expulsion or leakage of the dosage form to levels comparable to normal vaginal discharge.

The invention includes a method to formulate a mixture of synthetic cervical mucus and vaginal fluid suitable as a dosage form for vaginal administration that contains a therapeutic dose of UC-781. This dosage form is tested using the HIV-1 non-nucleoside reverse transcriptase inhibitor (NNRTI) UC-781 as a proof of principle. This approach of incorporating UC-781 into the formulation that mimics the resident fluids of the vagina should therefore provide optimal protection against HV-1 infection.

The synthetic mucus and vaginal formulations outlined herein are mixed in a 3 to 1 ratio as a reasonable estimate of in vivo conditions, and serve as the basis for the formulation effect. This mixture has a viscous fluid consistency (viscosity ˜3,000 to 5,000 cps). The appropriateness of this viscosity is determined after in vivo evaluations in humans have been completed. To allow for this uncertainty, three to four formulations are developed with a viscosity range from “viscous fluid” to “soft semisolid.” This is conducted by increasing the guar gum concentration in the synthetic mucus component.

In vivo studies with semi-solid formulations of UC-781 have been conducted that demonstrated appropriate antiviral activity at concentrations between 0.1% and 1%. Therefore, both 0.1% and 1% UC-781 will be tested in the formulations. However, lower concentrations, such as 0.001% UC-781, and any UC-781 concentration from at least about 0.001% to at least about 1.0% may also be tested. Accelerated stability studies are carried out to ensure the formulations maintain their physical and chemical integrity. Osmolarity, pH, viscosity and drug concentration will be the primary determinants of the formulation stability.

If an incompatibility between UC-781 and the polymer system in the synthetic cervical mucus formulation is observed, an investigation into other viscosity agents that can be crosslinked to provide the viscoelastic component exhibited in fresh mucus is conducted. This is unlikely since UC-781 is a non-polar, hydrophobic compound with limited solubility in water. Another potential consideration is the effect of semen on the physical integrity of the formulation. The addition of semen will change the component concentrations of the formulation, and could therefore potentially alter its physical properties. However, it is believed appropriately studying these effects is best achieved in vivo with established methods that rely on Magnetic Resonance Imaging (MRI) technology.

### Example 2

Determination of the Release Kinetics of UC-781 from the Formulation Matrix.

The release kinetic of UC-781 from the formulation matrix is determined utilizing a Franz diffusion cell apparatus following SUPAC guidelines for nonsterile semisolid dosage forms (Guidance for Industry, Nonsterile Semisolid Dosage Forms Scale-Up and post approval changes: Chemistry, Manufacture, and controls; In vitro release testing and in vivo bioequivalence documentation. U.S. Department of Health and Human Services, Food and Drug Administration Centers for Drug Evaluation and Resarch (CDER) May 1997 SUPAC-SS CMC 7).

This apparatus utilizes a membrane (synthetic or natural) to separate the formulation from the dissolution media. A suitable membrane (e.g. Teflon or cellulose acetate) and dissolution media (e.g. surfactant solution or phosphate buffered saline) is determined for this dosage form. Samples that are withdrawn from the receptor compartment are analyzed by HPLC to determine the concentration of drug released as a function of time.

The above methodology provides information on the movement of drug from the intact dosage form. It would also be desirable to know how the drug moves from the dosage form through a mucus/vaginal fluid layer encountered on actual vaginal administration. This is approximated in vitro by using a three-compartment diffusion cell containing a mucus/vaginal fluid layer between the donor compartment containing the formulation and the receiver compartment containing the dissolution media (Bhat et al., The Limiting Role of Mucus in Drug Absorption: Drug Permeation Through Mucus Solution, Int. J. Pharm. 126, 179-87 (1995)). The mucus/vaginal fluid layer will use mixtures of synthetic mucus/vaginal fluid of varying viscosities and fresh bovine and/or human cervical mucus/vaginal fluid.

### Example 3

Determination of the Biological Activity of the Formulation In Vitro Against HIV-1.

UC-781 is dispersed into the marketed vaginal product Replens® to be used as a control for all experimentation. The results of each test using the proposed synthetic vaginal fluid dosage form are compared to the results obtained for the control. Once successful, a delivery system for a wide variety of vaginal drugs, such as for example, hormones, anti-microbial agents and anti-HIV/STD drugs has been developed.

Once the dosage form has been developed and properly characterized physically and chemically, it is assessed in vitro for activity against HIV-1. The HIV-1 inhibitory effect of UC-781 is extremely potent (i.e., nanomolar range), and it is not expected that formulation of UC-781 in synthetic cervical mucus will have a significant effect on this activity. Unlike semi-solid gel or cream formulations, this dosage form is expected to have more rapid release kinetics. Thus, the drug should be readily available for interaction with HIV-1 virions or infected cells.

UC-781 activity has been evaluated using standard tissue culture methods (Buckheit, R. W., Snow, M. J., Fliakas-Boltz, V., et al., Highly potent oxathiin carboxanilide derivatives with efficacy against nonnucleoside reverse transcriptase inhibitor mutant human immunodeficiency virus isolates. Antimicro. Agents Chemo. 41:831-837, 1997)). For purposes of this study, primary isolates of HIV-1 are used to attempt infection of human PBMC in the presence of the formulation product. Specifically, the primary clade B isolates WeJo and 302056 are used, as well as other representative isolates form other non-clade B groups. The dosage formulation is sterilized by filtration and diluted prior to addition to PHA stimulated PBMC. Cultures are then challenged with the individual HIV-1 isolated at a final MOI (multiplicity of infection) in each test well of 0.1. After 7 days in culture at 37° C., 5.0% CO₂, supernatants are harvested for reverse transcriptase activity assessment. The IC₅₀ of the UC-781 formulation is calculated from the resulting titration curve as the concentration of required to obtain a 50% inhibitory effect on reverse transcriptase activity, relative to untreated control infected cultures.

### Example 4

Formulation of Microbicide and Detection of UC-781

Burruano, 2002 (supra) has produced a formulation for synthetic human cervical mucus and have extensively characterized the chemical, physical, and biological properties of UC-781. A vaginal simulant formulation (Owen and Katz, 1999; see above), and the synthetic cervical mucus formulation serve as the base for the microbicide formulation containing UC-781.

In order to appropriately detect UC-781, an isocratic HPLC method was developed that utilized UV detection at 297 nm in a 65:35 acetonitrile:water mobile phase, and has a limit quantitation of 0.1 μg/ml. This method is the basis of method development for analyzing UC-781 in the proposed formulation matrix.

### Example 5

Formulation and Manufacturing Process Optimization

The formulation will be manufactured at larger than lab scale to ensure it is a robust and reproducible process.

### Example 6

The UC-781 cervical mucus formulation is evaluated in an ex-vivo model system where human cervical or vaginal explant tissues are treated with the formulation and challenged by HIV-1 infection. In previous evaluations of UC-781 formulations, this model demonstrated that UC-781 remained active.

### Example 7

Evaluation of the Spreading, Distribution and Retention Properties of the Formulations in Human Volunteers.

This is achieved visually by mapping the movement of the formulation over time utilizing MRI. This is also conducted chemically by sampling the vaginal cavity of human volunteers after administration of the formulation and assaying the samples for drug content. Several points in the vaginal cavity are sampled and assayed with respect to time.

### Example 8

If the concept of designing a dosage form that mimics the resident fluids of the vagina is successful, other therapeutic agents will be incorporated into the formulation for vaginal delivery. A discussion of therapeutic agents envisioned as being useful in the practice of the invention is set forth above.

Further included in the invention are methods of using the formulation in the treatment, prevention and control of diseases or conditions affecting other mucus membranes such as the nose, throat and gastrointestinal tract, including the rectum.

Although the present invention as has been described in detail with reference to examples above, it is understood that various modifications can be made without departing from the spirit of the invention. All cited patents, patent applications and publications and other documents cited in this application are herein incorporated by reference in their entirety.

1. A method for delivering at least one therapeutic agent to a patient in need thereof comprising contacting patient mucosa with a formulation comprising:
(a) a composition selected from the group consisting of synthetic cervical mucus, synthetic vaginal fluid, and both synthetic cervical mucus and synthetic vaginal fluid, and,
(b) at least one therapeutic agent.

2. The method of claim 1, wherein the mucosa is vaginal or rectal.

3. The method of claim 1, wherein the patient is human and the therapeutic agent is N-[4-chloro-3-(3-methyl-2-butenyloxy)phenyl] (UC-781) or a derivative thereof.

4. The method of claim 3, wherein the UC-781 is present in the formulation from at least about 0.001% to at least about 1.0% wt %.

5. The method of claim 1, wherein at least one therapeutic agent is selected from the group consisting of hormones, anti-microbial agents, anti-viral agents, analgesic agents and anaesthetic agents.

6-7. (canceled)

8. The method of claim 1 wherein the synthetic vaginal fluid has at least two properties equal to, or substantially identical to, the properties of a composition comprising: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension.

9. The method of claim 1 wherein the synthetic vaginal fluid has the following composition: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of 4.2.

10. The method of claim 1, wherein the composition in step a) is synthetic cervical mucus comprising a viscosity which is optimal for delivery of at least one therapeutic agent.

11. The method of claim 1, wherein the viscosity of the synthetic cervical mucus is from about 2,000 cP to about 10,000 cP.

12. The method of claim 1, wherein the synthetic cervical mucus comprises guar gum present at about 1.00% w/w; and, dried gastric mucin (type III) present at about 0.50% w/w wherein the guar gum is cross-linked with borate.

13. The method of claim 12, wherein the synthetic cervical mucus further comprises imidurea, present at about 0.30% w/w; methylparaben, present at about 0.15% w/w; and, propylparaben, present at about 0.02% w/w.

14. The method of claim 13 wherein the synthetic cervical mucus further comprises dibasic potassium phosphate, present at about 0.26% w/w; and, monobasic potassium phosphate, present at about 1.57% w/w.

15. A method for treating or preventing a disease comprising contacting mucosa of a patient in need thereof with a formulation comprising:
(a) a composition comprising synthetic fluid suitable for vaginal use, and,
(b) an amount of at least one therapeutic agent effective to treat or prevent the disease.

16-21. (canceled)

22. The method of claim 15 wherein the synthetic fluid has at least two properties equal to, or substantially identical to, the properties of a composition comprising NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension.

23. The method of claim 15 wherein the synthetic fluid comprises one or more components selected from the group consisting of: NaCl, KOH, Ca(OH)₂ serum albumin, lactic acid, acetic acid, glycerol, urea and glucose.

24-28. (canceled)

29. A method for delivery of an effective amount of at least one therapeutic agent to a mucosal surface of a subject comprising administering a formulation to the mucosal surface wherein the formulation comprises guar gum present at about 1.00% w/w, dried gastric mucin (type III) present at about 0.50% w/w and a therapeutic agent present in an amount sufficient to be effective when the formulation is administered.

30-37. (canceled)

38. The method of claim 29 wherein the synthetic vaginal fluid has at least two properties equal to, or substantially identical to, the properties of a composition comprising: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of about 4.2; the properties selected from the group consisting of: pH, osmolarity and surface tension.

39. The method of claim 29 wherein the synthetic vaginal fluid has the following composition: NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L; wherein the composition has a pH of 4.2.

40-52. (canceled)

53. The method of claim 23 wherein the concentration of one or more components of the synthetic fluid is selected from the group consisting of NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L.

54. The method of claim 15 wherein the composition has a pH of 4.2.

55. The method of claim 15 wherein the synthetic fluid comprises NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L.

55. The method of claim 15 wherein the synthetic fluid consists of NaCl, 3.51 g/L; KOH, 1.40 g/L; Ca(OH)₂ 0.222 g/L; serum albumin, 0.018 g/L; lactic acid, 2.0 g/L; acetic acid, 1.0 g/L; glycerol, 0.16 g/L; urea 0.4 g/L; and glucose, 5.0 g/L.

56. The method of claim 15 wherein the therapeutic agent is is N-[4-chloro-3-(3-methyl-2-butenyloxy)phenyl] (UC-781) or a pharmaceutically acceptable salt thereof.

57. The method of claim 15 wherein the disease is a sexually transmitted disease.

58. The method of claim 57 wherein the sexually transmitted disease is transmitted by a virus.

59. The method of claim 58 wherein the virus is Human Immunodeficiency Virus (HIV).

60. The method of claim 15 wherein the disease is Acquired Immunodeficiency Syndrome (AIDS).
